(19) United States
(12) Patent Application Publication
(10) Pub. No.: US 2026/0272374 A1
(43) Pub. Date: Sep. 17, 2026

(54) SLEEP WELLNESS MANAGEMENT SYSTEM AND METHOD THEREOF

(71) Applicant: XUANWU HOSPITAL CAPITAL MEDICAL UNIVERSITY, Beijing (CN)

(72) Inventor: Hongxing WANG, Beijing (CN)

(21) Appl. No.: 19/166,319

(22) PCT Filed: Mar. 19, 2024
(86) PCT No.: PCT/CN2024/082513

§ 371 Date: Sep. 17, 2025

(30) Foreign Application Priority Data
Mar. 20, 2023 (CN) 202310271797.8
Mar. 20, 2023 (CN) 202310271798.2

(51) Int. Cl. A61B 5/00; A61B 5/369

(52) U.S. Cl. CPC A61B 5/4809; A61B 5/369

(57) ABSTRACT

A sleep wellness management system and a method thereof are provided, wherein the system includes a first sleep monitor and a second sleep monitor. The first sleep monitor collects at least one time-related first sleep characteristic datum from a user in a sleep state and determines a first sleep profile. The second sleep monitor collects at least one time-related second sleep characteristic datum from the user in the sleep state and determines a second sleep profile, and determines a difference between the first sleep profile and the second sleep profile, so as to generate an update information about a sleep metric of the first sleep monitor, thereby enabling the first sleep monitor to correct its monitoring result related to the first sleep profile according to the update information about the sleep metric. The present disclosure can enable portable wearable devices to achieve high-precision results of sleep monitoring.

This application is a National Stage application which claims priority from the international application PCT/CN2024/0082513, filed Mar. 19, 2024, which claims priority from CN application No. 202310271798.2, filed Mar. 20, 2023, which claims priority from CN application No. 202310271797.8, filed Mar. 20, 2023. The entirety of aforementioned application is incorporated herein by reference.

### BACKGROUND OF THE APPLICATION

### 1. Technical Field

The present disclosure relates to sleep monitoring, and more particularly to a sleep wellness management system and a method thereof.

### 2. Description of Related Art

Sleep can usually be characterized by two major modes, namely rapid eye movement (REM) sleep and non-REM sleep. REM sleep is also known as dreaming sleep. Non-REM sleep includes three stages that have different depth levels. NREM begins with the first and lightest NREM stage that happens right after falling asleep, then followed by the second NREM stage with relatively deeper but still light sleep, and ultimately the third NREM stage of deep sleep where it is very hard to wake the sleeper up. Sleep happens as a series of recurrent sleep stages, wherein the NREM and REM episodes alternate, and the whole process may be referred to as a sleep cycle.

As an example of sleep disorders, sleep apnea syndrome (SAS) features repeated occurrence of apnea, breath stoppage and/or low breathing during sleep. These conditions may last for seconds or even minutes. Carbon dioxide accumulates in the human body during apnea. Receptors in the blood streams sense the unduly high CO₂ level and signals the brain to wake up the sleeper for breath before the sleeper falls into sleep again. Similar scenarios may happen several times in one night and can terribly degrade the quality of sleep. In the long run, SAS can lead to various risks and fuel many health problems.

Hypopnea is another example of sleep disorders, and it appears as shallow breathing and abnormal respiratory rates. Usually, hypopnea is defined by decrease in air amount moving into the lungs. Just like SAS, hypopnea interferes with sleep seriously. For people with hypopnea, even they have slept for a whole night, they may nevertheless not feel rested.

Therefore, the quality of sleep during a sleep cycle is more than important to quality life. While sleep is obviously the key to our daily vitality, scientists are still working hard to understand what the “quality” and “quantity” of sleep humans need. Recently, great focus has been put on how sleep affects people physiologically and psychologically. For example, some may feel tired and weary in daytime if they did not sleep enough at night yet others may feel the same after they had a very long sleep.

For finding out the complex relationship and interaction between sleep and post-sleep vigor, researchers have made continuous investigation into various physiological conditions related to sleep, and as results, many sleep monitoring techniques have been proposed.

CN110882466B discloses a sleeping instrument, which comprises a main body and a magnetic coil electrically connected with the main body, wherein the main body comprises a main control device and a conversion circuit, the main control device is electrically connected with the magnetic coil, and the conversion circuit is electrically connected with the main control device; the main control device generates a square wave signal and inputs the square wave signal into the conversion circuit, the square wave signal is converted into a pulse signal through the conversion circuit, and the pulse signal is input into the magnetic coil so that the magnetic coil generates a time-varying magnetic field and couples the time-varying magnetic field to a brain sleep center of a patient with sleep disorder, thereby facilitating the adjustment and the induction of the sleep process of the patient and realizing the function of promoting sleep or awakening.

Most existing portable devices for sleep monitoring are designed to be wearable (e.g., watch-like smart sleep analyzers). They usually use an accelerometer to acquire motion data of users during sleep, and assess sleep quality of the users accordingly. However, since data of body motion as collected by an accelerometer are not directly related to sleep quality of users, the resulting assessment could be inaccurate. For example, a user who is sleeping may have involuntary movements and these movements reported by an accelerometer in a sleep-monitoring device may cause the device to wrongly determine that the user is not sleeping.

Sleep-monitoring instruments used in hospital usually analyze sleep quality based on electroencephalogram data. Electroencephalogram is known as the golden rule for defining sleep states, and existing electroencephalogram apparatuses for medical purposes are believed to be useful tools in such tasks. However, electroencephalogram signals may sometimes be quite weak, and instruments for accurately recording them thus have to be very precise and sensitive, making use of these costly and bulky apparatuses limited to healthcare institutions, like hospitals and clinics. Briefly, the existing professional apparatuses are not suitable for typical home use.

Hence, there is a need for a sleep monitoring apparatus/system that generates precise measurement results and provides non-professional users with direction or guidance for their home use, thereby helping its users to assess and improve sleep quality. There is also a need for a method/system that enables reliable monitoring or accurate quantification of sleep quality without using expensive sleep-monitoring instruments.

Since there is certainly discrepancy between the existing art comprehended by the applicant of this patent application and that known by the patent examiners and since there are many details and disclosures disclosed in literatures and patent documents that have been referred by the applicant during creation of the present disclosure not exhaustively recited here, it is to be noted that the present disclosure shall actually include technical features of all of these prior-art works, and the applicant reserves the right to supplement the application with the related art more existing technical features as support according to relevant regulations.

### SUMMARY OF THE APPLICATION

In view that existing portable sleep-monitoring devices are less capable for providing accurate monitoring results, the present disclosure uses regular calibration of sleep metric to enable a portable device to support accurate assessment of sleep profiles.

In view of the shortcomings of the existing art, according to a first aspect of the present disclosure, a sleep wellness management system is provided, aiming to solve at least one or more technical problems existing in the art known by the inventors.

To achieve the above objectives, the present disclosure suggests a sleep wellness management system which includes a first sleep monitor and a second sleep monitor. The first sleep monitor collects at least one time-related first sleep characteristic datum from a user who is in a sleep state and determines a first sleep profile. The second sleep monitor collects at least one time-related second sleep characteristic datum from the user who is in the sleep state and determines a second sleep profile, and then determines a difference between the first sleep profile and the second sleep profile, so as to generate an update information about a sleep metric of the first sleep monitor, thereby enabling the first sleep monitor to correct its monitoring result related to the first sleep profile according to the update information about the sleep metric. The present disclosure uses a second sleep monitor that accurately recognizes sleep profiles to update the sleep metric of a first sleep monitor, so that the first sleep monitor can maintain accurate in assessing sleep states of a user.

According to a preferred embodiment, the first sleep monitor and the second sleep monitor are connected to a terminal, respectively, in a wired and/or wireless manner, and the terminal retrieves a sleep-promoting regimen from a database according to the received second sleep profile. In the present disclosure, a terminal is introduced to analyze data, display data, and retrieve data, so that a user can be well informed of his/her sleep status and treatment programs.

According to a preferred embodiment, the first sleep monitor updates the sleep metric according to the received update information about the sleep metric so as to improve accuracy of the monitoring result related to the first sleep profile. Since the first sleep monitor is a portable/wearable device, inaccuracy may happen after long use. The present disclosure novelly ensures more accurate monitoring results of the first sleep monitor by updating sleep metric of the first sleep monitor.

According to a preferred embodiment, with the first sleep profile sent by the first sleep monitor, the terminal generates a sleep intervention datum based on the sleep-promoting regimen and the first sleep profile, so that a sleep intervention apparatus connected to the terminal performs a sleep intervention on the user according to the sleep intervention datum. The present disclosure hence provides a user with sleep intervention through the terminal and a sleep intervention apparatus, so that the sleep intervention apparatus can perform intervention appropriately, thereby improving sleep quality.

According to a preferred embodiment, the first sleep monitor collects the first sleep characteristic datum of the user through a wearable approach, and the second sleep monitor collects the second sleep characteristic datum of the user by collecting electroencephalogram signals of the user. As it is known that electroencephalogram data are reliable basis for analysis of sleep profiles, the present disclosure employs a second sleep monitor to calibrate the sleep metric of the first sleep monitor, thereby enabling the first sleep monitor to maintain accurate records of sleep.

According to a preferred embodiment, the second sleep monitor confirms that the first sleep profile and the second sleep profile are related to an identical sleep cycle in a time-based manner, and then determines the difference between the first sleep profile and the second sleep profile, and the second sleep monitor receives the sleep metric of the first sleep monitor, wherein the second sleep monitor adjusts the update information about the sleep metric of the first sleep monitor by taking the second sleep profile as the monitoring result that should be determined by that the first sleep monitor. The calibration of the sleep metric is based on data related to the same sleep cycle. This allows accurate determination of difference between two sleep profiles and prevents wrong calibration of the sleep metric.

According to a preferred embodiment, the update information about the sleep metric of the first sleep monitor includes one of the followings: a replacement information about the sleep metric and an adjustment difference of the sleep metric. The update information about each of the two sleep metrics can be used to calibrate or correct the sleep metric of the first sleep monitor, so as to ensure monitoring and determination of the sleep profile thereafter made by the first sleep monitor are accurate.

According to a preferred embodiment, the first sleep monitor includes a first collecting module, a first processing module and a first output module. The first collecting module acquires at least one first sleep characteristic datum that represents the sleep state of the user. The first processing module can determine the first sleep profile related to the user in response to the at least one first sleep characteristic datum acquired by the first collecting module. And the first output module outputs an information that is related to the first sleep profile and comes from the first processing module. In the present disclosure, the first sleep monitor can directly determine and output the first sleep profile according to a preset monitoring program.

According to a preferred embodiment, the second sleep monitor includes a second collecting module, a second processing module and a second output module. The second collecting module acquires at least one second sleep characteristic datum that represents the sleep state of the user, wherein the second sleep characteristic datum is different from the first sleep characteristic datum. The second processing module can determine the user-related second sleep profile in response to the at least one second sleep characteristic datum acquired by the second collecting module, and determine the difference between the first sleep profile and the second sleep profile to generate the update information about the sleep metric of the first sleep monitor. The second output module outputs the second sleep profile and the update information about the sleep metric of the first sleep monitor. The second sleep monitor is accurate in determining sleep profiles, and thus can be used for calibration and to generate the update information about the sleep metric.

In regard to a second aspect of the present disclosure, a sleep wellness management method is provided, wherein the method includes: having a first sleep monitor collect at least one time-related first sleep characteristic datum from a user who is in a sleep state and determining a first sleep profile; and having a second sleep monitor collect at least one time-related second sleep characteristic datum from the user who is in the sleep state, and determine a second sleep profile, so as to determine a difference between the first sleep profile and the second sleep profile, and output an update information about a sleep metric of the first sleep monitor, so that the first sleep monitor corrects its monitoring result related to the first sleep profile according to the update information about the sleep metric. In the disclosed sleep wellness management method, calibration of the first sleep monitor is simple and economic, and is therefore suitable for a user to regularly calibrate monitoring results of the first sleep monitor, thereby providing a reliable basis for sleep-promoting treatment.

According to a preferred embodiment, the method of the present disclosure further includes a step of: in the case where the second sleep monitor and a terminal are connected in a wired and/or wireless manner, having the terminal retrieve a sleep-promoting regimen from a database according to the received second sleep profile. The disclosed method supports relevant selection of promoting treatment regimen by achieving accurate determination of the sleep profile, so as to facilitate quality sleep interventions.

In a preferred embodiment of the inventive method, the first sleep monitor also updates the sleep metric according to the received update information about the sleep metric, so as to improve accuracy of the monitoring result related to the first sleep profile.

With respect to a third aspect of the present disclosure, a sleep monitoring system is provided, which includes a first sleep monitor and a second sleep monitor that are communicated with each other. The first sleep monitor and the second sleep monitor collect at least one time-related first sleep characteristic datum and at least one time-related second sleep characteristic datum that are related to a user in a sleep state, respectively, wherein the first sleep characteristic datum is different from the second sleep characteristic datum. The first sleep monitor determines a first sleep profile of the user according to the first sleep characteristic datum, and sends the first sleep profile to the second sleep monitor. And the second sleep monitor determines a second sleep profile of the user according to the second sleep characteristic datum, so as to determine a difference between the first sleep profile and the second sleep profile to generate an update information about a sleep metric of the first sleep monitor. Then the first sleep monitor receives the update information about the sleep metric and correspondingly corrects its monitoring result related to the first sleep profile.

The disclosed sleep monitoring system enables calibration of the first sleep monitor using merely interaction between two apparatuses, so as to ensure reliable sleep monitoring with minimal hardware requirements.

According to a preferred embodiment, an implementable sleep-promoting regimen that matches the first sleep monitor can be retrieved by the second sleep monitor from a database according to the second sleep profile, and with the first sleep monitor in communicative connection with a sleep intervention apparatus, the first sleep monitor sends a command information to the sleep intervention apparatus according to the first sleep profile of the user and the sleep-promoting regimen when the user is in a sleep state, so that the sleep intervention apparatus performs a sleep intervention according to the received command information. The sleep monitoring system of the present disclosure conducts sleep interventions using matching sleep-promoting regimens to improve sleep quality of a user.

As for a fourth aspect of the present disclosure, a sleep-promoting treatment terminal is provided, wherein the terminal can be in communicative connection with a first sleep monitor and a second sleep monitor, respectively, when a user is in a sleep state. The terminal is configured to receive an update information about a sleep metric of the first sleep monitor from the second sleep monitor, regularly calibrate the sleep metric of the first sleep monitor according to the update information about the sleep metric of the first sleep monitor, and receive a first sleep profile from the first sleep monitor and retrieve a relevant sleep-promoting regimen from a database.

The disclosed sleep-promoting treatment terminal is advantageous for it enables calibration of sleep metrics of a first sleep monitor with limited precision against monitoring data and sleep profile information from a high-precision second sleep monitor, and allows relevant selection of sleep-promoting regimens for the accurately determined first sleep profile, so as to provide a user with reasonable sleep intervention and in turn improve sleep quality of the user.

### BRIEF DESCRIPTION OF THE DRAWINGS

FIG. 1 is a structural diagram of a sleep promoting system according to a preferred mode of the present disclosure;

FIG. 2 illustrates the working logic of the inventive sleep wellness management system;

FIG. 3 illustrates the working logic of a terminal in the inventive sleep wellness management system; and

FIG. 4 is a flowchart of a sleep-promoting method according to a preferred mode of the present disclosure.

List of Reference Signs

| | |
|---|---|
| 100: First Sleep Monitor | 310: First Collecting Module |
| 200: Second Sleep Monitor | 320: Second Collecting Module |
| 300: Collecting Module | 410: First Processing Module |
| 420: Second Processing Module | |
| 510: First Output Module | |
| 520: Second Output Module | |
| 600: Terminal | |
| 700: Database | |
| 810: Extracting Unit | |
| 820: Analyzing Unit | |
| 830: Storing Unit | |

### DETAILED DESCRIPTION OF THE APPLICATION

The present disclosure will be described in detail with reference to the accompanying drawings.

### Embodiment 1

According to the American Academy of Sleep Medicine (AASM), a sleep cycle of an adult includes the following stages: W stage, also known as the stage of wakefulness; N1 stage, also known as the first stage of non-rapid eye movements (NREM1); N2 stage, also known as the second stage of non-rapid eye movements (NREM2); N3 stage, also known as the third stage of non-rapid eye movements (NREM3); and R stage, also known as the stage of rapid eye movements (REM). From W stage, the onset of sleep starts at NREM. NREM begins with N1 stage, which may take 3-7 minutes and is followed by N2 stage that may last for 10-25 minutes. Then N3 and N4 stages may happen in between several minutes and one hour. After deep sleep, N1 or N2 stage returns. Then the first REM sleep period starts and lasts for 5-10 minutes. This is the first sleep cycle. Specifically, in one sleep cycle, non-REM sleep takes up 75%-80%, and REM sleep takes up 20%-25%.

A typical adult may experience 4 to 6 sleep cycles each night. In the second half of overnight sleep, deep NREM sleep decreases gradually while REM sleep extends gradually. Specifically, in the first half of the night, N3 stage takes a large proportion, and as its proportion decreases in the second half of the night, REM increases.

Further, during sleep, the human brain can generate various electroencephalogram signals, such as α waves, β waves, θ waves, δ waves, and other non-characteristic waves, such as vertex sharp waves, spindle waves, and K-complex waves. Specifically, a waves are mainly seen under the eye-closed quiet alert state and in the R stage, at a frequency of 8-13 Hz. β waves are mainly seen in the eye-open alert state, being low-amplitude waves (typically 14-30 Hz) of a frequency>13 Hz. θ waves are mainly seen in late N1 stage, at a frequency of 4-7 Hz. δ waves are mainly seen in deep sleep, being 1-3 Hz low-frequency electrical waves. Therefore, electroencephalogram signals provide reliable information about different sleep profiles and complete sleep cycles a person experiences.

There are many portable sleep-monitoring devices (e.g., the first sleep monitor 100 as described herein) for home use. These devices are compact, light, easy to wear, and suitable for many scenes. Particularly, these sleep-monitoring devices measure the body temperature, the heart rate, and the respiratory rate, and thus provide sufficient information that well supports sleep monitoring at home. Besides, sleep-promoting functions (such as applying electric/magnetic stimulation, producing sleep-promoting neurotransmitters, or modulating related endocrines) provided by these sleep-monitoring devices are considered satisfying for daily sleep treatment tasks. However, as compared to medical-grade sleep monitors, these portable sleep-monitoring devices are inferior in precision and accuracy. This means one or more kinds of metrics provided by such a device over a sleep process, such as sleep cycles, sleep quality indexes, sleep efficiency, sleep onset latency, sleep fragmentation and/or other indicators, is likely to be inaccurate.

When a user uses such a portable sleep-monitoring device at home for self-measurement, the assessment results may have significant deviations, and this consequently leads to irrelevant sleep guidance and recommendations of sleep-promoting functions/measures. In this case, the portable sleep-monitoring device fails to provide accurate assessment, not to mention helping the user to take reasonable and effective means to get expected sleep wellness. In the worst case, the irrelevant sleep guidance and recommendations of sleep-promoting measures can even aggravate sleep troubles of a user by adding tiredness and disturbing biological rhythms, and eventually damage mental and physical health of the user.

To address the shortcomings of the existing portable/wearable devices about inaccurate monitoring results, failure in reaccommodating relevant sleep-promoting regimens, and incapability of providing effective sleep interventions through a sleep intervention apparatus, the present disclosure introduces the use of a high-precision sleep monitor so that a portable/wearable sleeping-monitoring device can have its sleep metric get calibrated or updated against data from the high-precision instrument, thereby achieving accurate determination and assessment of sleep profiles of users.

The embodiments and/or modes provided herein involve management systems that use user data, such as the heart rate, the body temperature, and the respiratory waveform, to monitor and quantify sleep quality of users, and methods thereof.

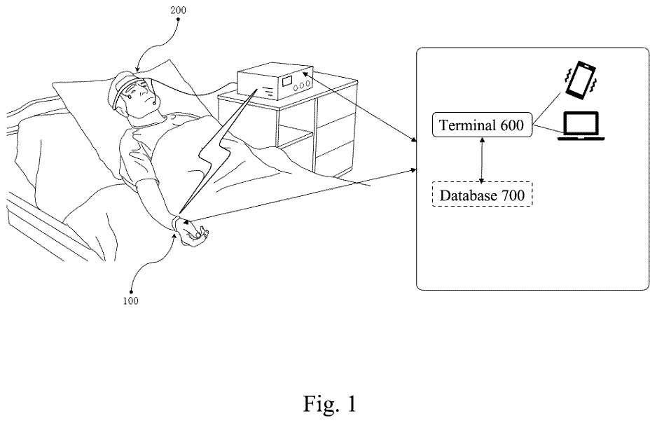

The present disclosure provides a sleep wellness management system. As shown in FIG. 1, the system comprises a first sleep monitor 100 and a second sleep monitor 200.

Specifically, the first sleep monitor 100 may be a wearable sleep monitor. The first sleep monitor 100 when worn by a user can collect a first sleep characteristic datum of the user. The wearable sleep monitor may be hand-held, watch-like, neck-mounted, or head-fit. Specially, in the present disclosure, the wearable first sleep monitor 100 may be in any other possible form, without limitation.

It has been proven that the physiological variations of a sleeping person are somehow related to the sleep state of that person. Based on this, a conventional sleep-monitoring instrument can determine the sleep state of a subject according to physiological signals of the subject, such as the body temperature, the heart rate, and the respiratory rate, as described previously. This includes determining the total sleep duration, the sleep stages corresponding to different time nodes, the segment duration of a certain sleep stage, the total lasting duration, stop points of and connecting points between different sleep stage, and sleep efficiency.

The first sleep characteristic datum may typically include signals indicating the body temperature (T), movement(S), the heart rate (H), and the respiratory rate (R). Specially, the signals of the body temperature (T), movement(S), the heart rate (H), and the respiratory rate (R) are stored in a time-related or frequency-related manner.

The first sleep monitor 100 is used for collecting at least one time-related first sleep characteristic datum from a user who is in a sleep state and determining a first sleep profile.

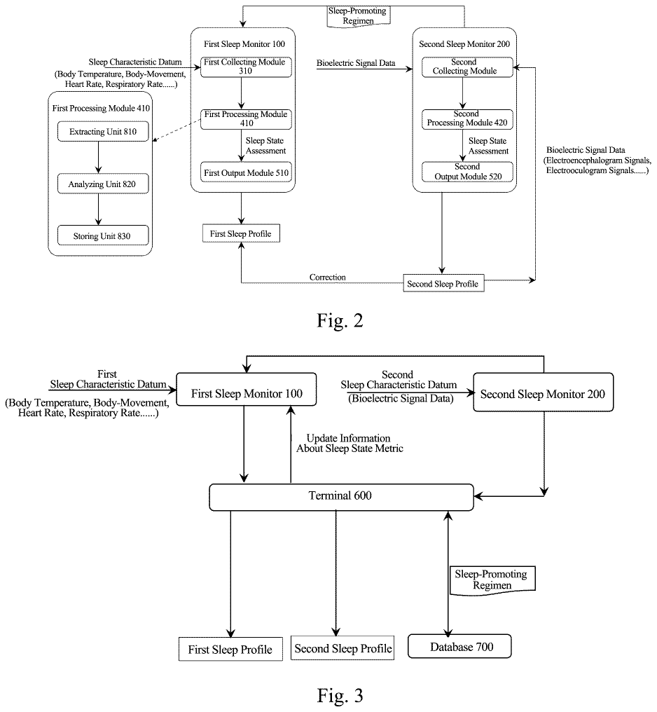

As shown in FIG. 2, the first sleep monitor 100 comprises a first collecting module 310, a first processing module 410, and a first output module 510. The first collecting module 310, the first processing module 410, and the first output module 510 are in communicative connection with each other in a wired and/or wireless manner.

The first collecting module 310 is used to acquire at least one first sleep characteristic datum that represents the sleep state of the user. The first collecting module 310 may be configured as a wearable device, a sensor or any other component that collects data related to the user. The first collecting module 310 may further have direct communication with a wearable device worn by the user, or it may acquire the first sleep characteristic datum of the user through a non-direct contact means (e.g., videos, IR, motion sensors, or other types of sensors).

In the present disclosure, the first collecting module 310 may be a sensor. Specifically, the first collecting module 310 may include one or more of the followings: a temperature sensor, a heart rate sensor, a respiratory sensor, and a motion sensor. The temperature sensor may be used to collect body temperature characteristic signals of the user. The heart rate sensor may be used to collect heart rate characteristic signals of the user. The respiratory sensor may be used to collect respiratory rate characteristic signals of the user. The motion sensor may be used to collect body-movement characteristic signal of the user.

The first processing module 410 can determine a first sleep profile related to the user in response to at least one first sleep characteristic datum acquired by the first collecting module 300.

The first processing module 410 has functions of analyzing and processing data. The first processing module 410 may be an ASIC chip, a CPU, or a processor that can execute the coded program of the first sleep profile. The first processing module 410 may comprise an extracting unit 810, an analyzing unit 820, and a storing unit 830. Specifically, the extracting unit 810 can extract one or more sleep characteristic data coming from the first collecting module 310. The analyzing unit 820 can store real-time sleep characteristic data in the storing unit 830 and analyze the first sleep state characteristic data according to the preset sleep metric so as to generate assessments results related to sleep profiles or sleep quality.

Preferably, the first processing module 410 is positioned outside the hardware body of the first sleep monitor 100, and can instruct the terminal 600 that is in communicative connection thereto to perform operation for determining the first sleep profile. This means that the first processing module 410 is replaced by the terminal 600. The terminal 600 feeds back the first sleep profile and the related information to the first sleep monitor 100.

The first output module 510 is used to output information related to the first sleep profile coming from the first processing module 410. The first output module 510 may output information about the sleep profile, time of the sleep profile, time periods, sleep cycles, and variations in sleep profiles.

Specially, the first output module 510 may output the sleep profiles of the user and the assessment information thereof through any means that communicates information, such as one or more of visual, haptic, acoustics and optical means. Alternatively, the generated first sleep profile and its assessment result may be provided through an interface. The interface may be a monitor, a mobile device, a laptop computer, a desktop computer, a wearable device, or a home computing device.

Specially, the sleep profiles may include sleep quality indexes, sleep cycles, total sleep duration, sleep efficiency, sleep onset latency, sleep fragmentation and/or other measurements. Further, the assessment results of the sleep profiles may be in the form of charts, curve graphs, texts, or any combination thereof.

In an example where the first collecting module 310 is a heart rate sensor, the extracting unit 810 can extract heart rate characteristic signals coming from the heart rate sensor. The analyzing unit 820 can store real-time heart rate characteristic signals into the storing unit 830 and analyze those signals to generate assessment results about the sleep state or sleep quality of the user. Further, the analyzing unit 820 can output the assessment results about the sleep profile or sleep quality of the user through the first output module 510 to the terminal 600 of the user or directly display the results. Specially, the heart rate sensor may be realized using one or more of the followings: an electrocardiogram sensor, a SaO2 sensor, an ultrasonic sensor, a photoplethysmography sensor and an RF sensor.

According to a preferred mode of the present disclosure, where the first collecting module 310 is a temperature sensor, the extracting unit 810 extracts body temperature characteristic signals from the temperature sensor. The analyzing unit 820 stores real-time body temperature characteristic signals into the storing unit 830 and analyzes those signals to get assessment results about the sleep profile or sleep quality of the user. Further, the analyzing unit 820 can output the assessment results of the sleep state or sleep quality of the user to the terminal 600 of the user or directly display them through the first output module 510.

According to a preferred mode of the present disclosure, where the first collecting module 310 is a respiratory sensor, the extracting unit 810 extracts respiratory rate characteristic signals from the respiratory sensor. The analyzing unit 820 stores real-time respiratory rate characteristic signals into the storing unit 830 and analyze those signals to get assessment results about the sleep state or sleep quality of the user. Further, analyzing unit 820 can output the assessment results about the sleep profile or sleep quality of the user to the terminal 600 of the user through the first output module 510 or display them directly to the user. Specially, the respiratory sensor may be realized using one or more of the followings: a displacement sensor, a strain sensor, and a photoplethysmography sensor.

Specially, in the present disclosure, the heart rate characteristic signals and the respiratory rate characteristic signals form cardiopulmonary characteristic signals of the user. Besides, the cardiopulmonary characteristic signals may include respiratory variation characteristic signals and cardiopulmonary coupling characteristic signals.

According to a preferred mode of the present disclosure, where the first collecting module 310 is a motion sensor, the extracting unit 810 extracts body-movement characteristic signals from the motion sensor. Specially, body-movement characteristic signals of a user may include characteristic signals indicating roll-over or twists of the user body. Further, the analyzing unit 820 stores real-time body-movement characteristic signal into the storing unit 830 and analyzes those signals to get assessment results about the sleep state or sleep quality of the user. Further, the analyzing unit 820 can output the assessment results about the sleep profile or sleep quality of the user to the user through the first output module 510. Specially, the body-motion sensor may be realized using one or more of the followings: a linear accelerometer, an angular accelerometer, and other sensors capable of detecting movements of objects.

According to a preferred mode of the present disclosure, after the first processing module 410 gets the assessment results about the sleep profile or sleep quality of the user, it can transmit these results same to an external electronic device, such as a computer or a smartphone, through an output interface for display or analysis. Alternatively, it may upload the results to a cloud database/server through the output interface so that the user can benefit from more comprehensive analysis and sleep guidance.

According to a preferred mode of the present disclosure, the first processing module 410 can determine the sleep profile or sleep quality of a user by analyzing plural sleep characteristic data of the user. Specifically, the first collecting module 310 can separately or simultaneously collect body temperature characteristic signals, heart rate characteristic signals, respiratory rate characteristic signals, and body-movement characteristic signals of the user. One or more extracting units 810 of the first processing module 410 can extract and amplify the body temperature characteristic signals, heart rate characteristic signals, respiratory rate characteristic signals, and/or body-movement characteristic signals of the user. The analyzing unit 820 can determine the sleep state or sleep quality of the user by analyzing and calculating the body temperature characteristic signals, heart rate characteristic signals, respiratory rate characteristic signals, and/or body-movement characteristic signals from the extracting unit 810.

In an optional mode of the present disclosure, the analyzing unit 820 can process one or more kinds of the sleep characteristic signals, such as the body temperature characteristic signals, heart rate characteristic signals, respiratory rate characteristic signals, and body-movement characteristic signals of the user, by means of weighted calculation, and analyze these sleep characteristic signals to generate assessment results about the sleep state or sleep quality of the user.

Specifically, a weighting coefficient may be assigned correspondingly to the body temperature characteristic signals, heart rate characteristic signals, respiratory rate characteristic signals, and body-movement characteristic signals of the user, respectively, and/or a weighting coefficient may be assigned correspondingly to the body temperature characteristic signal variation rate, heart rate characteristic signal variation rate, respiratory rate characteristic signal variation rate, and body-movement characteristic signal variation rate of the user, respectively. Afterward, based on a preset weighted average method, a sleep quality index related to the foregoing one or more sleep characteristic signal or characteristic signal variation rates can be obtained.

Further, the obtained sleep quality index may be compared with a preset quality threshold, and the gap or difference therebetween may be used to determine the sleep profile or sleep quality of a user. For example, if the sleep quality index is below the preset quality threshold, it is determined that the user is experiencing less-quality sleep.

Alternatively, in an optional mode of the present disclosure, the sleep characteristic signals of a user, such as the body temperature (T), movements(S), the heart rate (H), and the respiratory rate (R) may specifically be obtained as waveform diagrams, such as a body temperature waveform diagram, a movement waveform diagram, a heart-rate waveform diagram, and a respiratory waveform diagram. These waveforms may be acquired using a wearable sleep monitor.

According to a preferred mode of the present disclosure, after the extracting unit 810 acquires these waveforms of the sleep characteristic datum, the analyzing unit 820 divides these waveforms into plural segment waveforms. The analyzing unit 820 extract characteristic data from these segment waveform diagrams, such as body temperature data, heart rate data, or respiratory data. Further, the analyzing unit 820 can classify the sleep states or stages related to the user according to characteristic data in these segment waveform diagrams. Specially, the data model used by the analyzing unit 820 to perform classification of sleep profiles related to the user according to the characteristic data in the segment waveform diagrams may be obtained through machine learning, predetermined threshold programming, medical-staff custom, and/or any other possible means.

Specifically, the analyzing unit 820 divides sleep profile of the user into one or more stages of Period W, Period N1, Period N2, Period N3, and Period R. Further, the analyzing unit 820 may analyze one or more classes of sleep stages to obtain a result of the sleep profiles or sleep quality related to a user. For example, the result of the sleep profiles or sleep quality related to a user may be a sum of sleep quality indexes of different sleep stages. In addition, assessment results of sleep profiles or sleep quality may be determined according to one or more of the followings: total sleep duration, sleep efficiency, sleep onset latency, and sleep fragmentation.

According to a preferred mode of the present disclosure, the wearable first sleep monitor 100 may further be configured to apply/provide some waveform similar to electroencephalogram waves in frequency or a time-varying magnetic field prestored in the storing unit 830 for generating corresponding electroencephalogram waves to any affectable target area on a user's body. Specially, the affectable target area on a user's body may be an acupuncture point, such as Acupoint Baihui, Acupoint Shenfeng, Acupoint Zhaohai, or Acupoint Yongquan.

Specifically, when the analyzing unit 820 of the first sleep monitor 100 performs analysis according to one or more sleep characteristic data of the user during sleep acquired by the first collecting module 310 and determines that the sleep state or sleep quality of the user is not good or need to be improved, a sleep intervention apparatus may be used to perform sensory interventions on the user to change the sleep profile of the user. For example, where the sleep intervention apparatus is a magnetic field device, it may apply a time-varying magnetic field to the body of the user. Further, the time-varying magnetic field applied by the analyzing unit 820 through the magnetic field device is configured to be related to the sleep profile or sleep quality of the user.

The second sleep monitor 200 collects at least one time-related second sleep characteristic datum from the user who is in the sleep state and determining a second sleep profile. The second sleep monitor 200 collects a second sleep characteristic datum related to the user nu collecting electroencephalogram data related to the user. The second sleep monitor 200 compares the first sleep profile and the second sleep profile to determine a difference therebetween and generates an update information about the sleep metric of the first sleep monitor 100 accordingly, so that the first sleep monitor 100 can correct the monitoring results related to the first sleep profile according to the update information about the sleep metric.

The second sleep characteristic datum is different from the first sleep characteristic datum. The second sleep characteristic datum is bioelectric signal data. The bioelectric signal data may include electroencephalogram signals, electrooculogram signals, electromyogram signals, and electrocardiogram signals. Specially, the electroencephalogram signals, electrooculogram signals, electromyogram signals, and electrocardiogram signals are stored in a manner that they are related to time or frequency. Herein, the second sleep characteristic datum is for example in the form of electroencephalogram (EEG) signals.

In the present disclosure, the second sleep monitor 200 may be an electroencephalogram sleep monitor made to be used in hospitals or high-end medical clinics. Specifically, the second sleep monitor 200 may be, for example, a polysomnography system. Specially, the second sleep monitor 200 can determine sleep profiles of users at least according to electroencephalogram signals. Alternatively, the second sleep monitor 200 may determine the sleep profile of a user according to at least two forms of signal selected from electroencephalogram, electrooculogram and electromyogram. In other words, the second sleep monitor 200 determines the sleep profile of a user according to bioelectric signals.

According to a preferred mode of the present disclosure, the second sleep monitor 200 comprises a second collecting module 320, a second processing module 420, and a second output module 520. The second collecting module 320 is used to acquire at least one second sleep characteristic datum that represents the sleep state of a user. The second processing module 420 can determine a second sleep profile related to a user in response to at least one second sleep characteristic datum coming from the second collecting module 320. It then compares the first sleep profile and the second sleep profile to determine the difference therebetween and generates the update information about the sleep metric of the first sleep monitor 100. The second output module 520 is used to output the second sleep profile and the update information about the sleep metric of the first sleep monitor 100.

In the present disclosure, the first output module 510 and the second output module 520 may each be a display component, or a wired or wireless communication port.

Specifically, the second collecting module 320 (e.g., an electrode) of the second sleep monitor 200 is connected to the frontal pole of the user to collect electroencephalogram signals, electrooculogram signals, and electromyogram signals related to the user. The second collecting module 320 sends the electroencephalogram signals, electrooculogram signals, and electromyogram signals related to the user to the second processing module 420. The second processing module 420 comparatively analyzes the pre-processed electroencephalogram data by comparing with a sleep data model preloaded in the connected database 700, thereby determining the sleep profile or sleep quality of the user. The second output module 520 then outputs the sleep profile or sleep quality to the terminal 600 of the user or directly displays the information.

Generally speaking, existing sleep monitors like the second sleep monitor 200 typically employs bioelectric signals (e.g., electroencephalogram signals) to work. Since electroencephalogram has been scientifically recognized as the golden rule for assessing sleep wellness, medical-grade sleep monitoring equipment based on electroencephalogram is advantageous in view of its high detection precision and accuracy and extensively used in hospitals and high-end clinics. However, this is particularly inconvenient to people with disabilities because it requires a user to frequently visit the hospital or clinic for measurement and treatment. In addition to considerable time and efforts spent by users for the foregoing visits, a further disadvantage of the known approach is that the high costs for acquisition and maintenance the medical organization pays can be eventually passed to the user through his/her medical bill. Therefore, how to replace the existing bulky high-precision sleep-monitoring instruments with a wearable sleep-monitoring device that provides with consistent precision is an issue to address.

According to a preferred mode of the present disclosure, the second sleep monitor 200 determines the first sleep profile and the second sleep profile in the same sleep cycle in a time-based manner. For the same sleep cycle, the difference between the first sleep profile and the second sleep profile is determined through comparison. Where the second sleep monitor 200 and the first sleep monitor 100 are in direct or indirect interaction, the second sleep monitor 200 receives the sleep metric from the first sleep monitor 100. The second sleep monitor 200 adjust the update information about the sleep metric of the first sleep monitor 100 by taking the second sleep profile as the monitoring result that should be determined by the first sleep monitor 100.

Specifically, with the user in the sleep state, the first sleep monitor 100 and the second sleep monitor 200 are activated simultaneously and conduct synchronous monitoring for sleep profiles, respectively. After sleep monitoring is completed, the first sleep monitor 100 and the second sleep monitor 200 determines the first sleep profile and the second sleep profile, respectively. Where the two are in direct interaction, the first sleep monitor 100 sends the sleep profile and its sleep metric to the second sleep monitor 200. The second sleep monitor 200 compares the first sleep profile and the second sleep profile and their variations belonging the same sleep cycle, thereby determining the difference between the two profiles. The second sleep monitor 200 takes the second sleep profile as the correct monitoring result and adjusts the sleep metric of the first sleep monitor 100 against it, so that the first sleep monitor 100 can analyze the first sleep characteristic datum according to the new sleep metric and obtain the same monitoring result as the second sleep monitor 200 obtains.

In order to get more accurate sleep metrics, the second sleep monitor 200 uses differences between the first sleep profile and the second sleep profile over multiple sleep cycles to generate the update information about the sleep metric of the first sleep monitor 100. According to a preferred mode of the present disclosure, the update information about the sleep metric of the first sleep monitor 100 includes one of the followings: a replacement information about the sleep metric and an adjustment difference of the sleep metric. The replacement information about the sleep metric refers to a new sleep metric that replaces the old sleep metric, and is an updated sleep metric. The adjustment difference of the sleep metric refers to a difference value by which an adjustment shall be made to a sleep metric. As a result of adjusting the old sleep metric using the adjustment difference, a newly formed sleep metric is used to monitor and assess the first sleep state characteristic datum, so that the determined first sleep profile is identical or almost identical to the second sleep profile.

The second sleep monitor 200 sends the update information about the sleep metric to the first sleep monitor 100 directly or indirectly through the terminal 600. The first sleep monitor 100 updates the sleep metric according to the received update information about the sleep metric, thereby obtaining more accurate monitoring results related to the first sleep profile.

As described previously, the present disclosure enables a wearable sleep-monitoring device to provide precise and accurate results, so that a user can use such a wearable device to assess his/her sleep wellness at home conveniently and easily. The user can simply maintain accuracy of the first sleep monitor 100 by regularly calibrating its sleep metric against the second sleep monitor 200 in a hospital. Nevertheless, while the wearable device is improved to provide accurate monitoring results, the user may further need efficacious sleep-promoting regimens before he/she can have good sleep.

In order to recommend the user with a sleep-promoting regimen relevant to his/her determined sleep profile and implement the regimen to improve his/her sleep quality, or to protect him/her from danger, the disclosed system further comprises a terminal 600 and a sleep intervention apparatus, as shown in FIG. 1. The terminal 600 further serves to provide functions of data analysis to the first sleep monitor 100 and the second sleep monitor 200, thereby replacing the first processing module 410 and the second processing module 420. In an example where the terminal 600 is configured to execute a coded program for determining the first sleep profile, a coded program for determining the second sleep profile, and a coded program for analyzing the update information about the sleep metric preloaded thereto, the first sleep monitor 100 and the second sleep monitor 200 merely has to send the collected first and second sleep state characteristic data to the terminal 600 for determination of the sleep profiles. The first sleep monitor 100 receives the update information about the sleep metric fed back by the terminal 600 and uses it to update the sleep metric. This not only decreases processing loads of the first sleep monitor 100 and the second sleep monitor 200, but also reduces hardware requirements of the first sleep monitor 100 and the second sleep monitor 200. Thereby, the present disclosure is applicable to portable/wearable devices of different types or specifications or from different manufacturers for more accurate sleep metrics, and saves users money for device purchase.

According to a preferred mode of the present disclosure, as shown in FIG. 3, the first sleep monitor 100 and the second sleep monitor 200 are connected to a terminal 600 in a wired and/or wireless manner, respectively. The wired manner may involve building connection through a data cable, and the wireless manner may be achieved through a communication port, such as a Bluetooth-based communication component, an IR-based communication component, or a WiFi communication component. In an example where the terminal 600 is only communicatively connected to the first sleep monitor 100, the terminal 600 retrieves a relevant sleep-promoting regimen from the database 700 according to the received first sleep profile, as shown in FIG. 3. In an example where a terminal 600 is in communicative connection with the second sleep monitor 200, the terminal 600 retrieves a relevant sleep-promoting regimen from the database 700 according to the received second sleep profile.

According to a preferred mode of the present disclosure, with the first sleep profile received from the first sleep monitor 100, the terminal 600 generates a sleep intervention datum according to the sleep-promoting regimen and the first sleep profile, so that a sleep intervention apparatus connected to the terminal 600 can apply a sleep intervention on the user according to the sleep intervention datum.

Where the first sleep monitor 100 is in communicative connection with a sleep intervention apparatus, for a user who is sleeping, the first sleep monitor 100 sends a relevant command information to the sleep intervention apparatus according to the first sleep profile of the user and the sleep-promoting regimen, so that the sleep intervention apparatus can apply a sleep intervention according to the received command information.

According to a preferred mode of the present disclosure, sleep promotion may be achieved by applying low-frequency electromagnetic stimulation or digital-frequency-synthesis bionic electric waves to a subject. Specifically, when the portable sleep monitor determines that a subject has entered a light sleep stage, a hand-held or head-mounted sleep monitor may be used to apply a magnetic and/or electric field of a first frequency to the head or hand of the subject, so as to shorten the time before onset of deep sleep. Alternatively, when the portable sleep monitor determines that the subject has entered deep sleep, the hand-held or head-mounted sleep monitor may apply a magnetic and/or electric field of a second frequency to the head or hand of the subject, so as to extend this deep sleep. Depending on performance of different sleep electroencephalogram waveforms and other physiological indicators, sleep-promoting regimens corresponding to different sleep stages have different configuration parameters, such as the intensity, frequency, and duration of the electric current/magnetic field.

According to a preferred mode of the present disclosure, in an exemplary program where sleep promoting and monitoring instruments like the first sleep monitor 100 (e.g., a portable sleep-monitoring device) and/or the second sleep monitor 200 (e.g., medical-grade sleep-monitoring instrument) are used to help a subject with sleep, an alternating current may be introduced to stimulate sensory receptors of the subject, such as the median nerve, the radial nerve, and/or the ulnar nerve, so as to provide regulation onto a relevant encephalic region in the crania (e.g., the hypothalamus) through nervous sense, thereby mitigating sleeplessness.

As described previously, in order to overcome the shortcomings of existing portable sleep-monitoring devices that have been seen in home use scenarios, the present disclosure uses a highly precise and accurate medical-grade sleep-monitoring instrument to calibrate the sleep profile solely determined by a portable sleep-monitoring device. For example, one or more kinds of the metrics, such as sleep quality indexes, sleep efficiency, sleep onset latency, sleep fragmentation and/or other indicators, can be corrected so as to provide users with accurate and reliable sleep assessment.

Further, by using a medical-grade sleep-monitoring instrument to verify accuracy of the sleep profile determined by a portable sleep-monitoring device, the present disclosure effectively ensures and maintains high accuracy of a portable sleep-monitoring device for home use, and, according to the correctly determined sleep profile of a user, provides a reliable basis for selection of relevant sleep promoting measures, such as provision of a time-varying magnetic or electric field relevant to the sleep state of the user.

Specifically, for calibration, a subject visits a place equipped with a medical-grade sleep-monitoring instrument, such as a hospital or a high-end clinic, and wears both of the portable sleep-monitoring device (i.e., the first sleep monitor 100) and the medical-grade sleep-monitoring instrument (i.e., the second sleep monitor 200) for sleep monitoring. Preferably, the portable sleep-monitoring device and the medical-grade sleep-monitoring instrument are in signal communication, so that they can interact and share data.

A medical-grade sleep-monitoring instrument can use acquired bioelectric signals (e.g., electroencephalogram signals) to determine/acquire a second sleep profile related to a subject. Specially, to take advantage of the excellent precision of a medical-grade sleep-monitoring instrument, the terminal 600 may correct at least one sleep metric (e.g., the sleep quality index, sleep efficiency, or sleep onset latency) contained in the sleep profile determined by the portable sleep-monitoring device against data for at least another sleep cycle determined by a medical-grade sleep-monitoring instrument. Thereafter, when a subject uses only the portable sleep-monitoring device, for example at home, for sleep monitoring, the portable sleep-monitoring device will take the sleep metric in the same sleep cycle determined by the medical-grade sleep-monitoring instrument as the benchmark to correct data related to the current sleep profile, so as to achieve more accurate sleep assessment. In other words, the sleep metrics in the sleep assessment results acquired by the portable sleep-monitoring device are all corrected against the medical-grade sleep-monitoring instrument.

According to a preferred mode of the present disclosure, using the portable sleep-monitoring device and the medical-grade sleep-monitoring instrument to measure the sleep state of a subject so as to determine the corresponding sleep profile and using the sleep profile result obtained by the medical-grade sleep-monitoring instrument to correct one or more sleep assessment indicators determined by the portable sleep-monitoring device may involve having the subject experience one or more complete sleep cycles. The present disclosure may be used to find potential variations in sleep metrics of a subject under different sleep environments (including different scenes, physiological factors, and external interferences) in different sleep profiles and different sleep cycles.

Further, the sleep profile determined by the medical-grade sleep-monitoring instrument may be used to correct the sleep metrics in different sleep profiles and/or sleep cycles that are determined by the portable sleep-monitoring device, and the correction results may be fed back to and stored in the portable sleep-monitoring device, so as to improve the portable sleep-monitoring device in measurement accuracy. Additionally, precise sleep assessment provides valuable information for clarify the relationship between sleep quality and recovery of patients with sleep disorders and other diseases.

According to a preferred mode of the present disclosure, based on the differences in one or more sleep metrics between at least another sleep profile determined by the medical-grade sleep-monitoring instrument according to bioelectric signals and at least one sleep profile determined by the portable sleep-monitoring device, a controller can correct the sleep metrics in the sleep profile determined by the portable sleep-monitoring device against the relevant sleep metrics in the sleep profile determined by the medical-grade sleep-monitoring instrument, and transmit the corrected assessment results related to the sleep metrics to the portable sleep-monitoring device for storage. Specially, correcting the sleep metrics in the sleep profile determined by the portable sleep-monitoring device against the relevant sleep metrics in the sleep profile determined by the medical-grade sleep-monitoring instrument may be on the basis of preset correction program/algorithm, machine learning, or configuration by medical staff.

As sleep-monitoring devices and methods have been increasingly intelligent, with the sleep profile or sleep state determined for a given subject, either a portable sleep-monitoring device or a medical-grade sleep-monitoring instrument can formulate sleep-promoting regimens relevant to the sleep profile according to one or more sleep metrics contained in the determined sleep profile. Specifically, the sleep-promoting regimen may involve using a built-in magnetic field unit or an electrical stimulation unit to apply a magnetic or electric field that has impact on the sleep state of the subject. Specially, based on data from an accurate sleep assessment report of a subject, sleep-promoting regimens can be formulated to improve the sleep state of the subject. The sleep-promoting regimens may then be executed when appropriate to improve the sleep quality of the subject. As an extra and important advantage, these sleep-promoting regimen may help promote absorption of medication during sleep.

According to a preferred mode of the present disclosure, by calibrating the sleep monitoring result from a portable sleep monitor against the sleep monitoring result from a medical-grade sleep-monitoring instrument, formulating sleep-promoting regimens according to the corrected sleep monitoring result, and storing the regimens in the portable sleep monitor, a subject can use the portable sleep monitor to accomplish relevant and efficacious sleep-promoting treatment at home without frequently visiting a hospital or a high-end clinic for accessing a costly medical-grade sleep-monitoring instrument. Specially, these sleep-promoting regimens may be activated by the subject before he/she goes to bed or may be timed to activate. Alternatively, in the process that the portable sleep monitor performs real-time monitoring, these sleep-promoting regimens may be automatically activated in one or more certain sleep stages as set by the portable sleep monitor.

### Embodiment 2

The present embodiment provides further improvements on Embodiment 1, and repeated details are omitted from the description thereof.

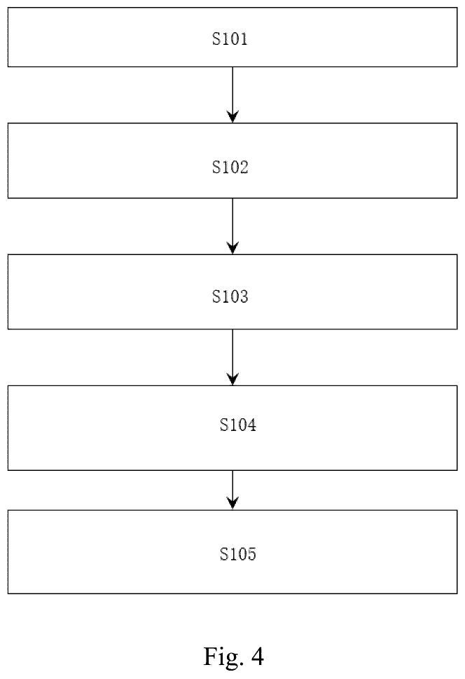

In a second aspect, the present disclosure provides a sleep wellness management method. As shown in FIG. 4, the method comprises the following steps:
S101: making a first sleep monitor 100 collect at least one time-related first sleep characteristic datum from a user who is in a sleep state and determining a first sleep profile;
S102: making a second sleep monitor 200 collect at least one time-related second sleep characteristic datum from the user who is in the sleep state and determining a second sleep profile;
S103: comparing the first sleep profile and the second sleep profile to determine a difference therebetween and outputting an update information about the sleep metric of the first sleep monitor 100 accordingly;
S104: making a first sleep monitor 100 correct monitoring results related to the first sleep profile according to the update information about the sleep metric; and
S105: with the second sleep monitor 200 or the first sleep monitor 100 connected to a terminal 600 in a wired and/or wireless manner, making the terminal 600 retrieve a relevant sleep-promoting regimen from a database 700 according to the received second sleep profile.

According to a preferred mode of the present disclosure, the method further comprises: making the first sleep monitor 100 update the sleep metric according to the received update information about the sleep metric, so as to improve accuracy of the monitoring results related to the first sleep profile.

### Embodiment 3

The present embodiment provides further improvements on Embodiment 1, and repeated details are omitted from the description thereof.

According to Embodiment 3, a sleep promoting system and an application method thereof are provided, aiming to solve at least one or more technical problems existing in the art known by the inventors.

To achieve the foregoing objective, the present disclosure provides a sleep promoting system. As shown in FIG. 1, it comprises a first sleep monitor 100 and a second sleep monitor 200 that interact with each other in terms of one or more sleep metrics related to a subject. Specially, the present disclosure facilitates sleep by helping users to, for example, fall asleep, enter deep sleep, and/or extend deep sleep, shorten transition between light and deep sleep, etc.

The first sleep monitor 100 is used to determine the first sleep profile according to one or more physiological characteristic data related to sleep duration of a subject, and apply an adjustable magnetic field/electric field related to sleep time or frequency of the subject onto an affectable target body part of the subject.

The second sleep monitor 200 is used to determine a second sleep profile that matches with the first sleep profile according to one or more physiological characteristic data related to sleep duration of the subject.

In the second sleep profile, the second sleep monitor 200 determines a third sleep profile according to one or more bioelectric signal data related to sleep duration of the subject, and corrects the at least one sleep metric contained in the first sleep profile according to the third sleep profile.

The second sleep monitor 200 then provides information about one or more sleep-promoting regimens related to the sleep stages of the user to the first sleep monitor 100 according to the one or more sleep metrics that are related to the first sleep profile and have been corrected according to the third sleep profile.

The first sleep monitor 100, in one or more determined sleep stages of the user, activates at least one sleep-promoting regimen that is related to the sleep stages and is determined according to one or more sleep metrics that are related to the first sleep profile and have been corrected according to the third sleep profile.

Preferably, the step of determining the sleep profile according to the one or more physiological characteristic data related to the sleep time or frequency of the subject comprises:
processing the data waveform of the one or more physiological characteristic data into a plurality of segment waveforms;
extracting one or more physiological characteristic data from each of the segment waveforms;
classifying the sleep stages of the user by the one or more physiological characteristic data in the segment waveforms; and
acquiring a sum of the sleep states of the user based on the classified sleep stages so as to determine the sleep profile.

Preferably, the physiological characteristic data include the body temperature, movements, the heart rate and/or the respiratory rate.

Preferably, the bioelectric signal data include electroencephalogram signals, electrooculogram signals, electromyogram signals, and/or electrocardiogram signals.

Preferably, the present disclosure further provides an application method for using the foregoing sleep promoting system. The application method comprises:
S201: through use of the first sleep monitor 100, determining the first sleep profile that corresponds to the one or more physiological characteristic data related to the sleep time or frequency of the subject;
S202: through use of the second sleep monitor 200, determining at least one second sleep profile that corresponds to the one or more physiological characteristic data related to the sleep time or frequency of the subject and matches with the first sleep profile;
S203: based on the second sleep profile, making the second sleep monitor 200 determine a third sleep profile corresponding to one or more bioelectric signal data related to the sleep time or frequency of the subject; and
S204: based on the third sleep profile, making the second sleep monitor 200 correct the one or more sleep metrics contained in the first sleep profile determined by the first sleep monitor.

Preferably, the application method based on the sleep promoting system of the present disclosure further comprises:
in one or more determined sleep stages of the user, making the first sleep monitor activate at least one sleep-promoting regimen that is related to the sleep stages of the user and is determined according to one or more sleep metrics corrected according to the third sleep profile and related to the first sleep profile.

### Embodiment 4

The present embodiment provides further improvements on Embodiment 1, and repeated details are omitted from the description thereof.

The present embodiment provides a sleep-promoting treatment terminal. When a user is in a sleep state, the terminal 600 is in communicative connection with the first sleep monitor 100 and the second sleep monitor 200, respectively.

The terminal 600 is configured to perform the following steps:
S301: receiving update information about a sleep metric of a first sleep monitor 100 from a second sleep monitor 200;
S302: based on the update information about the sleep metric of the first sleep monitor 100, regularly calibrating the sleep metric of the first sleep monitor 100; and
S303: receiving a first sleep profile from the first sleep monitor 100 and retrieving a relevant sleep-promoting regimen from a database 700.

The terminal 600 comprises one or more processors, memories, and communication buses used to at least connect the processors and the memories.

The memories are used to store one or more computer programs. Execution of the one or more computer programs by the one or more processors can cause the one or more processors to implement the application method of the sleep promoting system as disclosed herein.

According to a preferred mode of the present disclosure, the processor may include but is not limited to a central processing unit (CPU), a micro-processor unit (MPU), a micro control unit (MCU), or a System on Chip (SOC).

According to a preferred mode of the present disclosure, the memory may include but is not limited to a volatile memory (e.g., a DRAM or a SRAM) or a non-volatile memory (e.g., a FLASH, an optical disk, a soft drive, or a mechanical hard drive).

According to a preferred mode of the present disclosure, communication bus may include but is not limited to an ISA bus, a MAC bus, extended ISA, a VESA bus, or a PCI bus.

According to a preferred mode of the present disclosure, an electronic device in the context of the present disclosure may further include at least one communication interface. Specifically, the electronic device may be in communicative connection with at least one external apparatus through the communication interface. Moreover, the electronic device may be in communicative connection with at least one external network through a network interface card. The network interface card is in communicative connection with the communication bus.

The present embodiment further provides a storage medium that contains computer-executable commands. The computer-executable commands are to be executed by the computer processor to implement the operational steps of the sleep-promoting treatment terminal of the present disclosure.

According to a preferred mode, the computer storage medium of the present disclosure may be any combination of one or more computer-readable media. Each of the computer-readable media may be a computer-readable signal medium or a computer-readable storage medium. The computer-readable storage medium includes but is not limited to an electric, magnetic, optical, electromagnetic, infrared or semiconductor system, apparatus or device, or any combination thereof.

According to a preferred mode, more specific examples of the computer-readable storage medium include: electric connection with one or more cables, a portable computer disk, a hard drive, a random-access memory (RAM), a read-only memory (ROM), an erasable programmable read-only memory (EPROM or a flash memory), an optical fiber, a compact disc read-only memory (CD-ROM), an optical storage device, a magnetic storage device, or any combination thereof. In the present disclosure, the computer-readable storage medium may be any tangible medium containing or storing a program. The program may be used by or with a system, an apparatus or a device for executing instructions.

According to a preferred mode, the computer-readable signals medium may include data signals propagated in a baseband or as a part of carrier waves, in which computer-readable program codes are carried. Such propagated data signals may be in various forms, including but not limited to electromagnetic signals, optical signals, or any suitable combination thereof. The computer-readable signal medium may alternatively be any computer-readable medium other than a computer-readable storage medium. The computer-readable medium is able to send, propagate or transmit a program to be used by or with a system, an apparatus or a device for executing instructions.

According to a preferred mode, the program codes contained in the computer-readable medium may be transmitted using any suitable medium, including but not limited to wireless means, power cords, optical cables or RF, or any combination thereof.

According to a preferred mode, computer program codes used to execute the operation as described in the embodiments of the present disclosure may be written using one or more programming languages or a combination thereof. Suitable programming languages include object-oriented programming languages, such as Python, Java, Smalltalk, and C++, and include existing procedural programming languages, such as “C” language or similar programming language. The program codes may be completely executed in a user computer, partially executed in a user computer, executed as an independent software pack, partially executed in a user computer while partially executed in a remote computer, or completely executed in a remote computer or a server. In a case where a remote computer is involved, the remote computer may be connected to the user computer through a network of any type, such as a local area network (LAN) or a wide area network (WAN). Alternatively, it may be connected to an external computer (such as connected through the Internet by an Internet service provider).

It is to be noted that the particular embodiments described previously are exemplary. People skilled in the art, with inspiration from the disclosure of the present disclosure, would be able to devise various solutions, and all these solutions shall be regarded as a part of the disclosure and protected by the present disclosure. Further, people skilled in the art would appreciate that the descriptions and accompanying drawings provided herein are illustrative and form no limitation to any of the appended claims. The scope of the present disclosure is defined by the appended claims and equivalents thereof. The disclosure provided herein contains various inventive concepts, such of those described in sections led by terms or phrases like “preferably”, “according to one preferred mode” or “optionally”. Each of the inventive concepts represents an independent conception and the applicant reserves the right to file one or more divisional applications therefor.

What is claimed is:

1. A sleep wellness management system, comprising:
a first sleep monitor, for collecting at least one time-related first sleep characteristic datum from a user who is in a sleep state and determining a first sleep profile; and
a second sleep monitor, for collecting at least one time-related second sleep characteristic datum from the user who is in the sleep state and determining a second sleep profile, and determining a difference between the first sleep profile and the second sleep profile, so as to generate an update information about a sleep metric of the first sleep monitor, thereby enabling the first sleep monitor to correct its monitoring result related to the first sleep profile according to the update information about the sleep metric.

2. The sleep wellness management system of claim 1, wherein the first sleep monitor and the second sleep monitor are connected to a terminal, respectively, in a wired and/or wireless manner, and
the terminal retrieves a sleep-promoting regimen from a database according to the received second sleep profile.

3. The sleep wellness management system of claim 2, wherein the first sleep monitor updates the sleep metric according to the received update information about the sleep metric so as to improve accuracy of the monitoring result related to the first sleep profile.

4. The sleep wellness management system of claim 3, wherein with the first sleep profile sent by the first sleep monitor, the terminal generates a sleep intervention datum based on the sleep-promoting regimen and the first sleep profile, so that a sleep intervention apparatus connected to the terminal performs a sleep intervention on the user according to the sleep intervention datum.

5. The sleep wellness management system of claim 4, wherein the first sleep monitor collects the first sleep characteristic datum of the user through a wearable approach, and
the second sleep monitor collects the second sleep characteristic datum of the user by collecting electroencephalogram signals of the user.

6. The sleep wellness management system of claim 5, wherein the second sleep monitor confirms that the first sleep profile and the second sleep profile are related to an identical sleep cycle in a time-based manner, and then determines the difference between the first sleep profile and the second sleep profile, and
the second sleep monitor receives the sleep metric of the first sleep monitor,
wherein the second sleep monitor adjusts the update information about the sleep metric of the first sleep monitor by taking the second sleep profile as the monitoring result that should be determined by that the first sleep monitor.

7. The sleep wellness management system of claim 6, wherein the update information about the sleep metric of the first sleep monitor includes one of the followings: a replacement information about the sleep metric and an adjustment difference of the sleep metric.

8. The sleep wellness management system of claim 7, wherein the first sleep monitor comprises:
a first collecting module, for acquiring at least one first sleep characteristic datum that represents the sleep state of the user;
a first processing module, for determining the first sleep profile related to the user in response to the at least one first sleep characteristic datum acquired by the first collecting module; and
a first output module, for outputting an information that is related to the first sleep profile and comes from the first processing module.

9. The sleep wellness management system of claim 8, wherein the second sleep monitor comprises:
a second collecting module, for acquiring at least one second sleep characteristic datum that represents the sleep state of the user, wherein the second sleep characteristic datum is different from the first sleep characteristic datum;
a second processing module, for determining the user-related second sleep profile in response to the at least one second sleep characteristic datum acquired by the second collecting module; and determining the difference between the first sleep profile and the second sleep profile to generate the update information about the sleep metric of the first sleep monitor, and
a second output module, for outputting the second sleep profile and the update information about the sleep metric of the first sleep monitor.

10. A sleep wellness management method, comprising:
having a first sleep monitor-collect at least one time-related first sleep characteristic datum from a user who is in a sleep state and determining a first sleep profile; and
having a second sleep monitor collect at least one time-related second sleep characteristic datum from the user who is in the sleep state, and determine a second sleep profile, so as to determine a difference between the first sleep profile and the second sleep profile, and output an update information about a sleep metric of the first sleep monitor, so that the first sleep monitor corrects its monitoring result related to the first sleep profile according to the update information about the sleep metric.

11. The sleep wellness management method of claim 10, wherein
in the case where the second sleep monitor and a terminal are connected in a wired and/or wireless manner, the terminal retrieves a sleep-promoting regimen from a database according to the received second sleep profile.

12. The sleep wellness management method of claim 11, wherein
the first sleep monitor updates the sleep metric according to the received update information about the sleep metric so as to improve accuracy of the monitoring result related to the first sleep profile.

13. A sleep monitoring system, comprising a first sleep monitor and a second sleep monitor that are communicated with each other, wherein
the first sleep monitor and the second sleep monitor collect at least one time-related first sleep characteristic datum and at least one time-related second sleep characteristic datum that are related to a user in a sleep state, respectively, wherein the first sleep characteristic datum is different from the second sleep characteristic datum;
the first sleep monitor determines a first sleep profile of the user according to the first sleep characteristic datum, and sends the first sleep profile to the second sleep monitor;
the second sleep monitor determines a second sleep profile of the user according to the second sleep characteristic datum, so as to determine a difference between the first sleep profile and the second sleep profile to generate an update information about a sleep metric of the first sleep monitor, and
the first sleep monitor receives the update information about the sleep metric and correspondingly corrects its monitoring result related to the first sleep profile.

14. The sleep monitoring system of claim 13, wherein an implementable sleep-promoting regimen that matches the first sleep monitor can be retrieved by the second sleep monitor from a database according to the second sleep profile, and
with the first sleep monitor in communicative connection with a sleep intervention apparatus, the first sleep monitor sends a command information to the sleep intervention apparatus according to the first sleep profile of the user and the sleep-promoting regimen when the user is in a sleep state, so that the sleep intervention apparatus performs a sleep intervention according to the received command information.

15. (canceled)
